(19) United States
(12) Patent Application Publication
(10) Pub. No.: US 2013/0224755 A1
(43) Pub. Date: Aug. 29, 2013

(54) METHOD FOR DETECTING DNA ADDUCTS IN SALIVA

(71) Applicants: HAUH-JYUN CANDY CHEN, Chia-Yi (TW); Wen-Peng Lin, Yilan County (TW)

(72) Inventors: HAUH-JYUN CANDY CHEN, Chia-Yi (TW); Wen-Peng Lin, Yilan County (TW)

(73) Assignee: National Chung Cheng University, Chia-Yi (TW)

(21) Appl. No.: 13/599,246

(22) Filed: Aug. 30, 2012

(30) Foreign Application Priority Data
Feb. 29, 2012 (TW) 101106532

(51) Int. Cl. C12Q 1/68

(52) U.S. Cl. 435/6.14

(57) ABSTRACT

A method of detecting DNA adducts in saliva is disclosed herein. The method comprises the following steps: providing a salivary DNA; adding at least one isotope-labeled internal standard and a plurality of enzymes into the salivary DNA to hydrolyze the salivary DNA into a plurality of nucleosides; extracting the plurality of nucleosides by using a solid-phase extraction column; and detecting and quantifying at least one DNA adduct in the plurality of extracted nucleosides by utilizing a stable isotope dilution nano-flow liquid chromatography-nanospray ionization tandem mass spectrometry (LC-NSI/MS/MS).

### CROSS-REFERENCE TO RELATED APPLICATION

This application claims the benefit under 35 U.S.C. §119 of Taiwanese Patent Application No. 101106532, filed Feb. 29, 2012, which is hereby incorporated by reference in its entirety.

### BACKGROUND

1. Field of the Invention

The present invention relates to a detection method, and more particularly to a method of detecting DNA adducts in saliva.

2. Description of the Related Art

DNA adducts play an important role during a process of multistage cancer. However, since the quantity of DNA adducts is extremely few, how to analyze DNA adducts and clarify the role played by DNA adducts during cancer formation, progression and diagnosis is a great challenge. Clinical DNA samples are usually unable to obtain in mass except samples obtained from surgical operations. Therefore, an inspective method having high accuracy, high sensitivity and high specificity as a goal is required. To achieve the goal, liquid chromatography-mass spectrometry (LC/MS) is taken as a base methodology to directly analyze polar DNA adducts without derivatization. Recently, it has obvious advance in liquid chromatography interface and mass spectrometer ionization technique to improve its sensitivity. Since the sensitivity has been increased in analysis, the quantity of DNA samples required for analysis has been reduced as well. The foregoing situation is required for biological detection and research. To further increase the sensitivity of LC/MS, a capillary or nanoflow liquid chromatography is currently combined with nano-electrospray ionization MS or nanospray ionization MS (NSI MS) to have better effect in analysis of DNA adducts. Besides, a stable isotope substance that is equivalent to analyte structures is added in an experiment, which takes the mass spectrometer as basis, to compose an internal standard to accurately perform quantitative analysis for the analyte. Accordingly, errors caused by matrix effect or recovery of each step during the experimental process can be calibrated by the isotope-labeled internal standard. The most important thing is that since the trace amount of analyte can be easily lost in the experimental process having multiple steps, the isotope-labeled internal standard can be taken as a carrier for the trace amount of analyte in a biological sample to reduce the loss of the sample. Therefore, isotope dilution mass spectrometry has been widely applied in a low content DNA adduct of a living body requiring specific detection and accurate quantification.

A human is usually exposed to reactive α,β-unsaturated aldehyde, such as acrolein and crotonaldehyde, in environments of atmosphere, smokes or diesel exhaust gas. In addition, acrolein is also produced by cooking carbohydrate, oil and amino acid. The mutative exocyclic 1,N²-propano-2′-deoxyguanosine derived from acrolein (isomers of α-AdG and γ-AdG) and 1,N²-propano-2′-deoxyguanosine derived from crotonaldehyde (CdG) were generated through lipid peroxidation of acrolein and crotonaldehyde, respectively.¹⁻⁴ By comparing with normal people, the quantity of AdG in brain tissue DNA of Alzheimer disease patients is higher.⁵

Three etheno group adducts 1,N⁶-etheno-2′-deoxyadenosine, 3,N⁴-etheno-2′-deoxycytidine; εdCyd and 1,N²-etheno-2′-deoxyguanosine; 1,N²-εdGuo are mutative DNA lesions and are caused by externally industrial chemical or environmental pollution. In addition, these adducts are formed by lipid peroxidation in the body and play an important role at cancer formation, inflammation and atherosclerosis related to oxidation pressure.

In a former research, a nanoflow LC-NSI/MS/MS (liquid chromatography-nanospray ionization tandem mass spectrometry) has been developed and applied in a placenta or leukocyte DNAs of a human to respectively detect 1,N²-propano-2′-deoxyguanosine adduct and etheno group adduct in multiple reactions.⁶⁻⁷

Saliva is a DNA source that is quite easily obtained and has been currently used to detect related biological markers of oral cancer.⁸⁻¹⁰ DNAs separated from saliva mainly come from leukocyte or oral epithelial cells. The turnover time of oral epithelial cells is five to twelve days, and life time of leukocyte from gingival crevice is pretty short to express the body condition. Therefore, perhaps saliva can be taken as a detective biological marker having practicality and non-invasion as a tool of an intervention study for monitoring DNA damage inside the body related to carcinogen or oxidation pressure.

Consequently, a conventional method of detecting DNA adducts needs a lot of samples, obtains the samples through invasive manner and is unable to detect different kinds of adducts at the same time to cause labor force waste, material and time consuming. Therefore, an inventor of the invention designs a method of detecting DNA adducts in saliva to improve the conventional technique to further increase the implementation and utilization in industries.

### REFERENCES

(1) Chung, F. L.; Chen, H. J. C.; Nath, R. G. Carcinogenesis 1996, 17, 2105-2111.

(2) Yang, I.-Y.; Chan, G.; Miller, H.; Huang, Y.; Torres, M. C.; Johnson, F.; Moriya, M. Biochemistry 2002, 41, 13826-13832.

(3) Fernandes, P. H.; Kanuri, M.; Nechev, L. V.; Harris, T. M.; Lloyd, R. S. Environ. Mol. Mutagen. 2005, 45, 455-459.

(4) Stein, S.; Lao, Y.; Yang, I.; Hecht, S. S.; Moriya, M. Mutat. Res. 2006, 608, 1-7.

(5) Liu, X.; Lovell, M. A.; Lynn, B. C. Anal. Chem. 2005, 77, 5982-5989.

(6) Chen, H.-J. C.; Lin, W.-P. Anal. Chem. 2009, 81, 9812-9818.

(7) Chen, H.-J. C.; Lin, G.-J.; Lin, W.-P. Anal. Chem. 2010, 82, 4486-4493.

(8) Walsh, D. J.; Corey, A. C.; Cotton, R. W.; Forman, L.; Herrin, G. L., Jr.; Word, C. J.; Garner, D. D. J. Forensic Sci. 1992, 37, 387-395.

(9) Lum, A.; Le Marchand, L. Cancer Epidemiol. Biomarkers Prev. 1998, 7, 719-724.

(10) Prasad, M. P.; Mukundan, M. A.; Krishnaswamy, K. Eur. J. Cancer B: Oral Oncol. 1995, 31B, 155-159.

### BRIEF SUMMARY

In view of the shortcomings of the prior art, the inventor(s) of the present invention based on years of experience in the related industry to conduct extensive researches and experiments, and finally developed a method of detecting DNA adducts in saliva as a principle objective to overcome defects of conventionally requiring large amount of samples and obtaining samples through invasive manners and that may not detect different kinds of adducts at the same time to waste labor forces, materials and experimental time.

To achieve the foregoing object, a method of detecting DNA adducts in saliva is provided and comprises steps of providing a salivary DNA (deoxyribonucleic acid); adding at least one isotope-labeled internal standard and a plurality of enzymes into the salivary DNA so that the salivary DNA is hydrolyzed into a plurality of nucleosides; extracting the nucleosides by utilizing a solid-phase extraction column; and detecting and quantifying at least one DNA adduct in the plurality of extracted nucleosides by utilizing a stable isotope dilution nanoflow liquid chromatography-nanospray ionization tandem mass spectrometry (LC-NSI/MS/MS).

Preferably, the at least one DNA adduct may be one of 1,N²-propano-2′-deoxyguanosine, including 1,N²-propano-2′-deoxyguanosine isomers derived from acrolein (α-AdG and γ-AdG); 1,N²-propano-2′-deoxyguanosine derived from crotonaldehyde (CdG); 1,N⁶-etheno-2′-deoxyadenosine (εdAdo); 3,N⁴-etheno-2′-deoxycytidine (εdCyd); 1,N²-etheno-2′-deoxyguanosine (1,N²-εdGuo); or a combination thereof.

Preferably, the quantification limit of the DNA adducts AdG, CdG, εAdo, εCyd and 1,N²-εdGuo may be respectively 0.1 picogram, 0.5 picogram, 0.1 picogram, 0.5 picogram and 0.5 picogram.

Preferably, the isotope-labeled internal standard may be [¹⁵N₅]AdG, [¹⁵N₅]CdG, [¹⁵N₅]εdAdo, [¹⁵N₃]εdCyd or [¹³C₁,¹⁵N₂]1,N²-εdGuo.

Preferably, the enzymes may include micrococcal nuclease, phosphodiesterase II, adenosine deaminase and alkaline phosphatase.

Preferably, the salivary DNA may be at least 25 microgram.

Preferably, a spray voltage of the stable isotope dilution nanoflow LC-NSI/MS/MS may be 1.3 to 2.0 kV.

Preferably, a source temperature of the stable isotope dilution nanoflow LC-NSI/MS/MS may be 200 to 300 degrees Celsius.

Preferably, an analysis mode of the stable isotope dilution nanoflow LC-NSI/MS/MS may be a high selective reaction mode (H-SRM).

The method of detecting DNA adducts in saliva according to the invention has one or more advantages as the following:

(1) The detection method of the invention obtains samples through non-invasive manners to easily acquire the samples.

(2) The detection method of the invention can detect different kinds of DNA adducts at the same time to decrease examination time, reduce the consumption of operational labor forces and reduce the material waste.

(3) The detection method of the invention is highly sensitive and merely needs few samples to complete the measurements.

### BRIEF DESCRIPTION OF THE DRAWINGS

FIG. 1 is a structural diagram of DNA adducts detected by the detecting method according to the invention.

FIG. 2 is a flowchart according to an embodiment of the invention.

FIG. 3 is a chromatogram of detecting human salivary DNA with internal standards according to an embodiment of the invention.

### DETAILED DESCRIPTION

The foregoing and other technical characteristics of the present invention will become apparent with the detailed description of the preferred embodiments and the illustration of the related drawings.

The method of detecting DNA adducts in saliva according to the invention can utilize saliva as a sample to simultaneously detect more than one of DNA adducts and take an embodiment as the following.

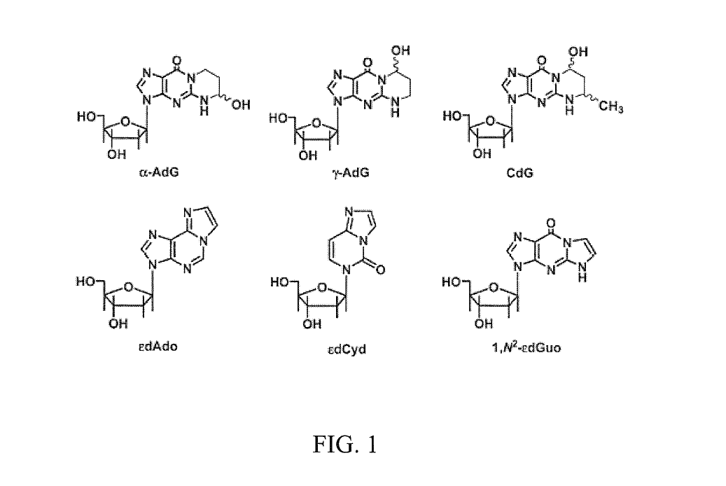

With reference to FIG. 1 for a structural diagram of a method of detecting DNA adducts according to the invention is depicted. As shown in the figure, AdG (α-AdG and γ-AdG), CdG, εdAdo, εdCyd and 1,N²-εdGuo are outer ring DNA adducts.

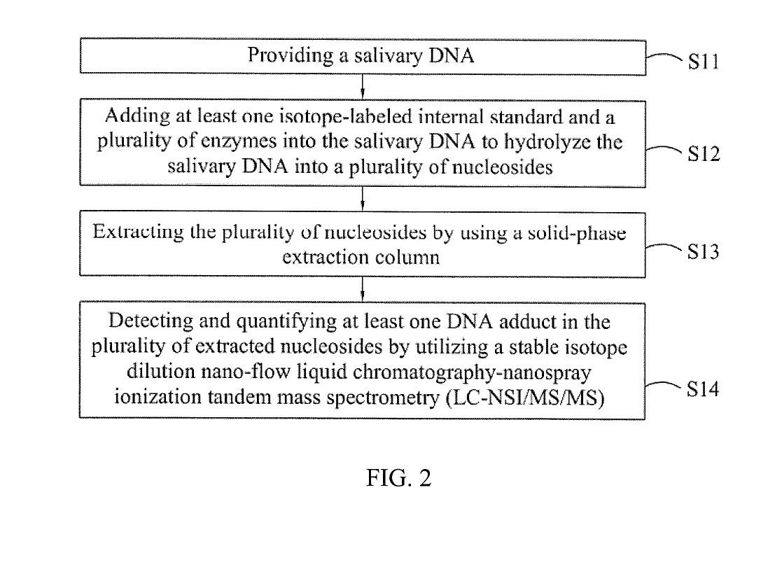

With reference to FIG. 2 for a flowchart according to an embodiment of the invention is depicted. S11: providing a salivary DNA; S12: adding at least one isotope-labeled internal standard and a plurality of enzymes into the salivary DNA to hydrolyze the salivary DNA into a plurality of nucleosides; S13: extracting the plurality of nucleosides by using a solid-phase extraction column; and S14: detecting and quantifying at least one DNA adduct in the plurality of extracted nucleosides by utilizing a stable isotope dilution nano-flow liquid chromatography-nanospray ionization tandem mass spectrometry (LC-NSI/MS/MS).

Experimental method:

### Human Salivary DNA Isolation

In one embodiment of the invention, the sample comes from healthy volunteers. These volunteers receive a written guarantee to depict that data of all volunteers is merely for research purpose, and personal information of these volunteers is confidential. The volunteers are required to fasting for one hour before collecting saliva. Before collecting saliva, the volunteers use tooth paste to brush their tooth and rinse the mouths thoroughly. Blood DNA extraction midiprep systems (Viogen, Sunnyvale, Calif.) are utilized to extract slivary DNA from collected saliva according to the instruction of the specification. The steps are simply depicted as the following: three milliliter extraction buffer is added into per three milliliter saliva, and 25 milligram/milliliter proteinase K is added into the mixture. The mixture then is vibrated for 20 seconds and placed with incubation at 60 degrees Celsius for 84 minutes and further placed at 70 degrees Celsius for 56 minutes. During the placing term, the mixture is vibrated once every 7 minutes. Three milliliter ethanol having 99.5% concentration is added into the mixture that is further vibrated. Afterward the mixture is added in a genomic DNA midi column, and the column is performed with 2500 g centrifugal force for 3 minutes. Filtrate is drained out, and wash buffer is used to wash a precipitate in the column three times. The column is performed with 4900 g centrifugal force for 5 minutes to dry the column. Three milliliter pure water at 70 degrees Celsius is added in the column, and after placing the column at 70 degrees Celsius for 5 minutes, the column is performed with 2500 g centrifugal force for 10 minutes to elute the salivary DNA in the column. The repeat elution is performed three times to achieve the optimum salivary DNA productivity.

A nano-drop 1000 photometer (J&H Technology CO., Ltd., Wilmington, Del.) is utilized to quantify the extracted salivary DNA. The purity of salivary DNA is checked by utilizing A260/A280 absorbance ratio. The ratio is between 1.7 and 2.0 to perform subsequent experiments. The production of entire extraction process is 9.3±4.5 microgram DNA (average±standard deviation) extracted from per milliliter saliva. The range is 2.4 to 20 microgram/milliliter. With respect to the sample having fewer DNA content, more saliva is collected.

### Enzyme Hydrolysis of DNA

In an embodiment of the invention, the solution containing 25 microgram salivary DNA is added into the adducts [¹⁵N₅]AdG, [¹⁵N₅]CdG, [¹⁵N₅]εdAdo, [¹⁵N₃]εdCyd, and [¹³C₁, ¹⁵N₂]1,N²-εdGuo containing each 100 picogram isotope-labeled. The mixture is performed with enzyme hydrolysis through the following enzyme hydrolysis manners A or B. Preferably, only 5 micrograms DNA is used to simultaneously analyze and quantify signals and contents of five adducts while in real analysis.

### Hydrolysis Manner A

In an embodiment of the invention, the solution containing 25 micrograms salivary DNA and five isotope-labeled internal standards is dissolved into 10 millimole concentration sodium succinate/5 millimole concentration CaCl₂ buffer (pH7.0), and the mixture then is placed at 100 degrees Celsius for 30 minutes. Next, the mixture is cooled down to reach a room temperature. Three units micrococcal nuclease; from Staphylococus aureus and 0.01 unit phosphodiesterase II; from bovine spleen are added into the mixture, and the mixture is placed at 37 degrees Celsius for 6 hours to perform enzyme hydrolysis. Next, 0.9 unit adenosine deaminase; from bovine spleen and 6 units alkaline phosphatase; from calf intestine are added into the mixture, and the mixture is placed at 37 degrees Celsius for overnight.

### Hydrolysis Manner B

In an embodiment of the invention, the the solution containing 25 micrograms salivary DNA and five isotope-labeled internal standards is dissolved into 50 millimole concentration ammonium acetate buffer (pH5.3), and 2.4 units nuclease P1 is added into the mixture. The mixture then is placed at 45 degrees Celsius for two hours. Tris-HCl buffer; pH7.4 and 0.0024 unit phosphodiesterase I; from Crotalus adamanteus venom are added into the mixture, and the mixture is placed at 37 degrees Celsius for two hours to neutralize the enzyme hydrolysis reaction. Finally, 0.9 unit adenosine deaminase; from bovine spleen and 0.6 unit alkaline phosphatase; from calf intestine are added into the mixture, and the mixture is placed at 37 degrees Celsius for one hour.

### Adducts Enrichment

In an embodiment of the invention, the hydrolysate is filtered by 0.22 micrometer nylon syringe filter. Next, after the solid-phase extraction column (SPE; Bond Elut C18, 100 mg, 1 mL, Varin; Harbor City, Calif.) is performed with rinsing and balancing through methyl alcohol and water, the solid-phase extraction column is utilized to purify a plurality of DNA adducts existing in a form of nucleoside. Next, after the solid-phase extraction column is sequentially washed by three milliliter water and one milliliter aqueous solution containing methyl alcohol (5%), one milliliter aqueous solution (25%) containing methyl alcohol can be added to collect the hydrolysis product. After drying the hydrolysis product and re-dossolving the product in 10 microliter acetic acid; 0.1%, 0.22 micrometer nylon syringe filter is utilized to re-filter the mixture. Finally, a treated 2 microliter sample is extracted to perform nanoflow LC-NSI/MS/MS analysis as the following.

### Stable Isotope Dilution Nanoflow LC-NSI/MS/MS (Liquid Chromatography-Nanospray Ionization Tandem Mass Spectrometry) Analysis

In an embodiment of the invention, a 2 microliter injection loop is connected to a 6-port switching valve of a liquid chromatography system, wherein the liquid chromatography system is composed of an UltiMate 3000 nano LC system (Dionex, Amsterdam, Netherlands) and a reversed phase tip column (75 μm×130 mm, 5 μm; MAGIC C18AQ, 200 Å, 5 μm; Michrom BioResource, Auburn, Calif.). The pum out (30 microliter/minute) is divided into a 300 nanoliter/minute of flow rate. The moving phase A is 1% acetic acid while the moving phase B is acetonitrile containing 0.1% acetic acid to set a start a linear gradient from 5% moving phase B to 30% moving phase B within 5 minutes. The effluent is analyzed by a triple quadrupole mass spectrometer (TSQ Quantum Ultra EMR mass spectrometer; Thermo Electron Corp., San Jose, Calif.) having a nano-spray ionic interface. The column effluent enters the spray chamber through a tapered emitter constructed by a 75 micrometer fused-silicon capillary and directly electrosprayed into the spectrometer under the positive ion mode of the nano-electrospray ionic cascaded spectometer. The spray can be monitored by a built-in charge coupled device (CCD) camera. The voltage of spray is 1.6 kV, and the source temperature is 220 degrees Celsius. Argon gas is taken as collision gas during the experiment of the spectrometer.

With the sample concentrated by the adducts that is analyzed by nanoflow LC-NSI/MS/MS, parent ions [M+H]⁺ ionized in a quadrupole post Q1 and the collision cell Q2 are converted to generate product ions. The product ions in a third quardrupole Q3 are further analyzed by utilizing high selective reaction monitoring; H-SRM mode, wherein the mass width of the Q1 and the Q3 is 0.2 m/z and 0.7 m/z respectively, and a dwell time is 0.1 second. Ions selected from the Q1 and the Q3, and the collision energy at the Q2 is listed in table 1, wherein the Q1 of multiple reaction monitoring (MRM) condition 2 is equivalent to the Q1 of multiple reaction monitoring (MRM) condition 1. For example, product ions of AdG, εdAdo and 1,N²-εdGu are [M+H−116]⁺ ([M+H−dR]⁺ ions. The product ions of CdG and εdCyd are respective [M+H−160]⁺ ([M+H−dR−C₂H₄O]⁺) and [M+H−171]⁺ ([M+H−dR−CO−HCN]⁺) ions.

TABLE 1

| | Multiple reaction | | | Multiple reaction | |
|---|---|---|---|---|---|
| | monitoring | | | monitoring | |
| | condition 1 (MRM) | | | condition 2 (MRM) | |
| | | | Collision | | Collision |
| | | | energy | | energy |
| | Q1 | Q3 | (eV) | Q3 | (eV) |
| AdG | 324.0 | 207.8 | 15 | 164.0 | 35 |
| [¹⁵N₅]AdG | 329.0 | 212.8 | 15 | 169.0 | 35 |
| CdG | 338.0 | 178.0 | 35 | | |
| [¹⁵N₅]CdG | 343.0 | 183.0 | 35 | | |
| εdAdo | 276.1 | 160.0 | 20 | 119.1 | 45 |
| [¹⁵N₅]εdAdo | 281.1 | 165.0 | 20 | 123.1 | 45 |
| εdCyd | 252.1 | 81.0 | 45 | 108.1 | 35 |
| [¹⁵N₃]εdCyd | 255.1 | 83.0 | 45 | 111.1 | 35 |
| 1,N²-εdGuo | 292.1 | 176.0 | 25 | 148.1 | 35 |
| [¹³C₁,15N₂]1,N²-εdGuo | 295.1 | 179.0 | 25 | 151.1 | 35 |

### Statistic Analysis

GraphPad InStat version 3.00 (GraphPad Software, San Diego, Calif.) is used to perform statistic analysis. Related coefficients between contents of any two adducts are calculated by utilizing Pearson linear correlation.

### Result and Discussion

### Establishment of Experimental Method

After teeth of a salivary donor are completely brushed, saliva is collected in a cleaned container. Salivary DNA is extracted by the extraction kit for extracting blood DNA and the extraction method. After performing quantification for extracted salivary DNA through a spectrophotometer, the yield of obtaining salivary DNA is 2.4 to 20 microgram DNA/per milliliter saliva. The yield is similar to the foregoing reported manner. The foregoing DNA solution is added with the following five isotope-labeled internal standard, including [¹⁵N₅]AdG, [¹⁵N₅]CdG, [¹⁵N₅]εdAdo, [¹⁵N₃]εdCyd and [¹³C₁,¹⁵N₂]1,N²-εdGuo. The mixture is performed with enzyme hydrolysis. Next, the adducts, which have been treated with enzyme hydrolysis, are concentrated by utilizing a reversed-phase solid-phase extraction column and are analyzed under the highly selective reaction monitoring (H-SRM) mode by utilizing nanoflow LC-NSI/MS/MS analysis. The optimum collision energy and highly selective reaction monitoring transition are shown in the condition 1 of multiple reactions monitoring of table 1. In addition to CdG and εdCyd, great majority is proptonated base adduct ions after daughter ions in molecular ions lose deoxyriboxe moiety. The proptonated base adduct ions of CdG and εdCyd in the chromatograph have the existence of disruptors, and it will affect the accuracy of quantification. Therefore, other daughter ions are additionally searched as the condition of H-SRM. The detection limit of five adducts is similar to the foregoing documents. In addition, since α-AdG and γ-AdG are isomers, structures are extremely similar such that in the analysis system, both chromatography signals are not separated, and the content of AdG on quantification is the sum of α-AdG and γ-AdG.

### Enzyme Hydrolysis

In the foregoing research, the quantity and categories of hydrolytic enzyme and pH value and standing time of hydrolysis reaction greatly influence the hydrolysis of DNA adducts. In an embodiment of the invention, the optimum hydrolysis manner A of AdG and CdG within human salivary DNA is compared with the optimum hydrolysis manner B of three kinds of etheno adducts within human salivary DNA, and the result is shown in table 2. The hydrolysis manner A can release AdG at five times and 1,N²-εdGuo at two times more than the hydrolysis manner B. In addition, the quantities of CdG and εdCyd released by the hydrolysis manner A and the hydrolysis manner B are similar. On the other hand, 18% εdAdo can be released by the hydrolysis manner B by comparing with the hydrolysis manner A. Such result is not a surprise. The hydrolysis manner B is originally to hydrolyze εdAdo. In an embodiment of the invention, since the sensivity of detecting εdAdo through the hydrolysis manner A is higher, and the detection limit further reaches 0.73 mole that is the highest in five analyzed adducts, the hydrolysis manner A is selected to perform subsequent experiments.

TABLE 2

Average adduct (adducts/10⁸ nucleotide) ±
standard deviation

| Adducts | Hydrolysis manner A | Hydrolysis manner B |
|---|---|---|
| AdG | 218 ± 11 | 43.4 ± 3.6 |
| CdG | 6.33 ± 0.43 | 5.83 ± 0.56 |
| εdAdo | 99.2 ± 3.8 | 117 ± 2 |
| εdCyd | 67.7 ± 6.2 | 66.1 ± 2.7 |
| 1,N²-εdGuo | 672 ± 11 | 333 ± 9 |

### Method Validation

Calibration curves are constructed by drawing on the chromatogram of the liquid chromatography mass spectrometry. In another word, the ratio of the peak area of isotope-labeled internal standard (100 picogram) to the peak area of adducts (0.1 to 300 picogram) is obtained to form the curves. After the drawing, the related coefficients (R²) of five adducts AdG, CdG, εdAdo, εdCyd and 1,N²-εdGuo are respectively 0.9997, 0.9992, 0.9999, 0.9996 and 0.9999. The lowest quantity of analyte of the calibration curves is also the limit of quantification in the embodiment of the invention. The limit of quantification of five adducts AdG, CdG, εdAdo, εdCyd and 1,N²-εdGuo are repsecively 0.1 picogram (0.31 femtomole), 0.5 picogram (1.5 femtomole), 0.1 picogram (0.36 femtomole), 0.5 picogram (2.0 femtomole), and 0.5 picogram (1.7 femtomole). In a control experiment, signals of adducts are unable to be detected if an isotope is merely added or an isotope and hydrolytic enzyme are simultaneously added. It represents that none disruptor and pollutant exist in the chromatogram of hydrolytic enzyme or internal standard.

In an embodiment of the invention, three kinds of known adduct standards are added into human placenta DNA (25 microgram), and the total amount of the adducts is measured to confirm the accuracy of the detection method according to the invention. With the extrapolation of the linearly regressed lines, the quantities of five adducts AdG, CdG, εdAdo, εdCyd and 1,N²-εdGuo are respectively 55.2, 6.8, 17.3, 10.5 and 27.0 picogram. The quantities of the adducts measured in DNA without standard are respectively 57.6, 7.7, 16.3, 9.7 and 26.3 picogram in the five adducts AdG, CdG, εdAdo, εdCyd and 1,N²-εdGuo. These results represent that the detection method of the invention has the excellent accuracy and quality control regardless of adding standard or not. The quantity of adducts within DNA can be correctly measured.

In an embodiment of the invention, the accuracy of the detection method according to the invention is evaluated by utilizing two sets of human salivary DNAs. Each sample is repeated three times every day and performed with repeat motion within three different days. The result is shown in table 3. The relative standard deviation (% RSD) of five adducts of two sets of samples within intraday is 0.7% to 9.7%, and the % RSD everyday is 2.9% to 9.1%.

TABLE 3

| Average adduct (adducts/10⁸ nucleotide) ± | | | |
|---|---|---|---|
| standard deviation | | | |
| Day 1 | Day 2 | Day 3 | Day deviation |

No. 26

| | | | | |
|---|---|---|---|---|
| AdG | 108 ± 3.8 | 111 ± 0.9 | 111 ± 0.5 | 110 ± 3.0 |
| CdG | 3.4 ± 0.2 | 3.9 ± 0.2 | 3.3 ± 0.07 | 3.6 ± 0.32 |
| εdAdo | 111 ± 0.8 | 121 ± 2.8 | 119 ± 4.0 | 117 ± 5.6 |
| εdCyd | 70.7 ± 6.6 | 70.3 ± 2.5 | 68.1 ± 4.8 | 69.7 ± 4.7 |
| 1,N²-εd | 456 ± 44 | 441 ± 17 | 432 ± 9.8 | 443 ± 26 |

No. 27

| | | | | |
|---|---|---|---|---|
| AdG | 156 ± 4.2 | 163 ± 2.6 | 163 ± 3.8 | 161 ± 5.4 |
| CdG | 9.4 ± 0.1 | 9.7 ± 0.2 | 9.1 ± 0.2 | 9.4 ± 0.4 |
| εdAdo | 219 ± 4.9 | 221 ± 1.7 | 210 ± 3.9 | 219 ± 4.9 |
| εdCyd | 118 ± 5.4 | 109 ± 6.6 | 106 ± 1.0 | 118 ± 5.4 |
| 1,N²-εdGuo | 518 ± 41 | 543 ± 4.2 | 536 ± 12 | 519 ± 41 |

In an embodiment of the invention, two sets of different human salivary DNAs are analyzed by utilizing multiple reaction monitoring transitions. The quantity of the adducts is further validated except CdG. The result is shown in table 4. Under the multiple reaction monitoring condition 2, although it has lower sensitivity except εdCyd, remained adducts still use higher collision energy. The calibration curve of the multiple reaction monitoring condition 2 also has great linearity. The relative coefficients of four adducts AdG, εdAdo, εdCyd and 1,N²-εdGuo are respectively 0.9982, 0.9999, 0.9994 and 0.9994. By utilizing different multiple monitoring conditions, identical results can be obtained in two sets of different human salivary DNAs, and the beneficial evidence that has no artificial contaminant is also provided for the quantitative result in the embodiment.

TABLE 4
Average adducts (adducts/10⁸ nucleotide) ± standard deviation

| | Sample No. 26 | | Sample No. 27 | |
|---|---|---|---|---|
| | Multiple | Multiple | Multiple | Multiple |
| | reaction | reaction | reaction | reaction |
| | monitoring | monitoring | monitoring | monitoring |
| | condition 1 | condition 2 | condition 1 | condition 2 |
| AdG | 111 ± 0.5 | 106 ± 0.3 | 163 ± 3.8 | 145 ± 3.6 |
| εdAdo | 119 ± 4.0 | 128 ± 1.2 | 210 ± 3.9 | 195 ± 4.4 |
| εdCyd | 68.1 ± 4.8 | 60.4 ± 0.6 | 106 ± 1.0 | 102 ± 3.3 |
| 1,N²-εdGuo | 432 ± 9.8 | 448 ± 10 | 536 ± 12 | 523 ± 20 |

While individually detecting salivary DNA and adding no internal standards, the interference of internal standards is not found in salivary DNA. However, it must be careful that artificial adducts may be produced during the sample operation and/or analysis process. Perhaps glutathione can be added in the reaction to inhibit artificial adducts from being produced. However, glutathione that affects DNA extraction and enzyme within enzymatic hydrolysis must be evaluated before performing the experiment.

### The Quantity of Adducts in Human Salivary DNA

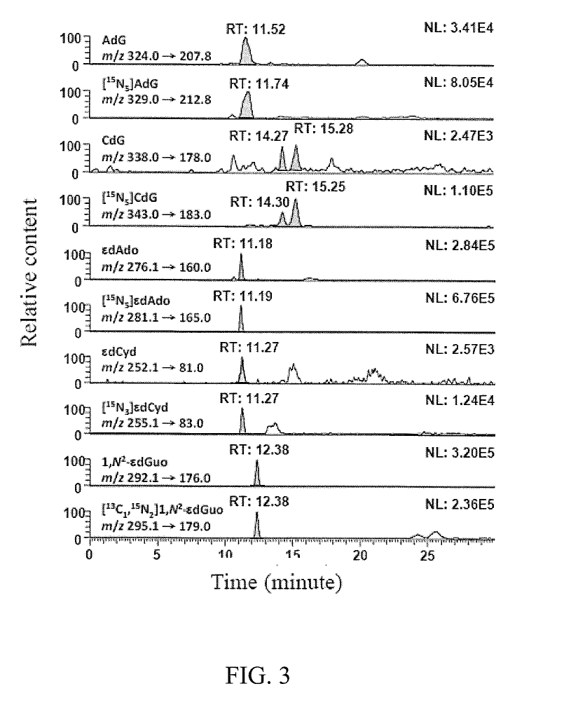

In an embodiment of the invention, salivary DNAs of 27 healthy volunteers are analyzed. These volunteers are not exposed to industrial chemicals during working. With reference to FIG. 3 for a chromatogram of human salivary DNA added with internal standards according to an embodiment of the invention is depicted. The diagram is the chromatograms of nanoflow LC-NSI/MS/MS of highly selective reaction monitoring transitions with respect to the isotope-labeled internal standards and five adducts AdG, CdG, εdAdo, εdCyd and 1,N²-εdGuo. Under the reaction condition, five adducts elute out prior to 18 minutes. The system depicted by the prior arts needs 25 minutes to elute AdG and CdG out. The quantities of five adducts AdG, CdG, εdAdo, εdCyd and 1,N²-εdGuo are respectively 152, 9.21, 223, 122 and 489/108 normal nucleotide. Table 5 concludes the quantities of five adducts within salivary DNAs of 27 volunteers. The reproducibility of the detection method according to the invention is represented by the average of % RSD. In three repeated experiments, the average relative standard deviation of adduct is 4.5%, and the range of the % RSD is 0.05% to 12%.

TABLE 5

| | Average adduct (adducts/10⁸ nucleotide) ± | | | | |
|---|---|---|---|---|---|
| | standard deviation | | | | |
| sample | AdG | CdG | εdAdo | εdCyd | 1,N²-εdGuo |
| 1 | 13 ± 1 | ND | 75.0 ± 3.3 | 40.5 ± 3.5 | 68.4 ± 2.4 |
| 2 | 74 ± 1 | 2.51 ± 0.07 | 26.0 ± 1.7 | ND | 106 ± 0.9 |
| 3 | 35 ± 2 | 4.7 ± 0.4 | 55.2 ± 5.5 | 98.6 ± 1.4 | 162 ± 0.2 |
| 4 | 32 ± 0.6 | ND | 31.2 ± 1.4 | 43.6 ± 5.1 | 144 ± 0.07 |
| 5 | 85 ± 1.9 | ND | 129 ± 1.4 | 107 ± 8 | 548 ± 11 |
| 6 | 155 ± 8 | 2.26 ± 0.05 | 100 ± 0.3 | 77.8 ± 6 | 560 ± 20 |
| 7 | 167 ± 4 | 1.55 ± 0.04 | 90.4 ± 1.8 | 95.7 ± 4.4 | 365 ± 3 |
| 8 | 132 ± 4.7 | 6.4 ± 0.27 | 153 ± 2.5 | 139 ± 10 | 752 ± 12 |
| 9 | 176 ± 19 | 16.7 ± 0.6 | 73.6 ± 3.0 | 49.5 ± 4.3 | 286 ± 4.7 |
| 10 | 108 ± 9 | 5.22 ± 0.44 | 81.2 ± 4.0 | 52.4 ± 4.9 | 290 ± 6.6 |
| 11 | 116 ± 3 | ND | 210 ± 3.1 | 229 ± 3.3 | 589 ± 5 |
| 12 | 158 ± 6 | 4.8 ± 0.2 | 155 ± 4.3 | 99.5 ± 1.2 | 634 ± 7 |
| 13 | 106 ± 1 | 1.1 ± 0.1 | 72.2 ± 0.8 | 10.8 ± 0.1 | 627 ± 17 |
| 14 | 108 ± 2 | 48.5 ± 3.0 | 65.6 ± 0.6 | 48.9 ± 2.8 | 269 ± 4 |
| 15 | 117 ± 3 | 4.0 ± 0.2 | 83.8 ± 1.7 | 25.2 ± 0.8 | 374 ± 2.1 |
| 16 | 114 ± 2 | 6.7 ± 0.4 | 63.1 ± 1.1 | 29.7 ± 1.5 | 233 ± 7 |
| 17 | 106 ± 8 | 19.0 ± 0.8 | 69.4 ± 0.6 | 51.5 ± 3.3 | 322 ± 6 |
| 18 | 81.5 ± 6.6 | 5.5 ± 0.4 | 113 ± 3 | 120 ± 11 | 670 ± 12 |
| 19 | 139 ± 2 | 43.0 ± 3.0 | 130 ± 6 | 105 ± 1.5 | 343 ± 7 |
| 20 | 81.7 ± 6.9 | 1.3 ± 0.1 | 94.3 ± 0.6 | 46.8 ± 3.8 | 377 ± 9 |
| 21 | 63.2 ± 3.4 | 2.8 ± 0.2 | 131 ± 7 | 44.4 ± 1.8 | 356 ± 17 |
| 22 | 66.2 ± 0.8 | 2.5 ± 0.1 | 173 ± 9 | 134 ± 15 | 466 ± 20 |
| 23 | 22.8 ± 0.3 | ND | 22.1 ± 1.6 | 11.6 ± 0.6 | 72.3 ± 3.5 |
| 24 | 65.5 ± 2.6 | 4.5 ± 0.1 | 49.0 ± 2.8 | 43.6 ± 0.8 | 397 ± 7 |
| 25 | 218 ± 11 | 6.3 ± 0.4 | 99.2 ± 3.8 | 67.7 ± 6.2 | 672 ± 11 |
| 26 | 111 ± 0.5 | 3.3 ± 0.07 | 119 ± 4.0 | 68.1 ± 4.8 | 432 ± 9.8 |
| 27 | 163 ± 3.8 | 9.1 ± 0.2 | 210 ± 3.9 | 106 ± 1.0 | 536 ± 12 |
| Aver- | 104 ± 50 | 7.5 ± 12 | 99 ± 50 | 72 ± 49 | 391 ± 198 |
| age ± | | | | | |
| stan- | | | | | |
| dard | | | | | |
| devia- | | | | | |
| tion | | | | | |
| Range | 13-218 | 0-48.5 | 22-210 | 0-139 | 68-752 |

### Correlation Between Individual Adduct in Human Salivary DNA

In an embodiment of the invention, the correlation between any two selected adducts is analyzed by utilizing Pearson linear regression. The result is shown in table 6 and represents that the quantity of AdG obviously correlates to the quantity of 1,N²-εdGuo. The correlation coefficient (γ) is 0.5756 (p<0.0001) but does not correlate to CdG or εdCyd. In a etheno group adduct, the quantity of εdAdo and the quantity of εdCyd have obvious correlation extremely. The correlation coefficient (γ) is 0.8007 (p=0.0017). The quantity of εdAdo and the quantity of 1,N²-εdGuo have obvious correlation, and the correlation coefficient (γ) is 0.6778 (p=0.0001). The correlation coefficient (γ) of εdCyd and 1,N²-εdGuo is 0.5643 (p=0.0022). The correlation coefficient (γ) of AdG and εdAdo is 0.3969 (p=0.0404). These results represent that three etheno group adducts may come from the same sources such as endogenous lipid peroxidation.

TABLE 6

| | AdG | CdG | εdAdo | εdCyd | 1,N²-εdGuo |
|---|---|---|---|---|---|
| AdG | — | 0.2588 | 0.3969 | 0.2414 | 0.5756 |
| | | (0.1923) | (0.0404) | (0.2250) | (0.0017) |
| CdG | — | — | −0.1859 | −0.0073 | −0.9825 |
| | | | (0.9267) | (0.9707) | (0.6259) |
| εdAdo | — | — | — | 0.8007 | 0.6778 |
| | | | | (<0.0001) | (0.0001) |
| εdCyd | — | — | — | — | 0.5643 |
| | | | | | (0.0022) |
| 1,N²-εdGuo | — | — | — | — | — |

The relatively higher correlation coefficient (γ=0.8007) between εdAdo and εdCyd might be hinted that the quantity of adduct could react with the quantity of another adduct. Therefore, perhaps two adducts are unnecessary to be simultaneously analyzed in the future. However, normal people may preferably analyze εdAdo not εdCyd since the detection limits of analyzing εdAdo and εdCyd using nanoflow LC-NSI/MS/MS are respectively 0.73×10⁻¹⁸ mole and 160×10⁻¹⁸ mole. It is obviously more sensitive in the detection of εdAdo than εdCyd.

Contrarily, CdG and other four adducts do not have statistically obvious correlation. This might represent that sources of CdG and other four adducts are different. CdG is not only derived from crotonaldehyde but also derived from acetaldehyde and is a metabolite of alcohol and carbohydrate and is also an environmental pollutant.

The quantity of adducts in tissue DNAs usually represents the balance between the production of adducts and adducts removed by repair enzyme and is different in each tissue. For example, εdAdo and εdCyd are measured by utilizing ³²P tagging method. The result has obvious difference in smooth muscle cells within abdominal aortas of atherosclerosis patients but does not have obvious difference in pancreas within smokers and non-smokers.

Another reason for the inconsistent correlation between quantities of adducts might be the individual's pathological or physiological status, resulting in the occurrence of adduction reactions at livers, colons, pancreas and stomachs. In addition, people who are exposed to cigarette, oil-smokes and intake of fatty acid, vegetables, vitamins E and alcohol may have different influence on the quantity of adducts.

Utilizing saliva to diagnose diseases of cardio-vascular disease, breast cancer and oral cancer is quite practical. The formal research demonstrates that 8-oxodG in plasma, saliva and urine may correlate with diseases. If urine is not extracted with DNA, DNA adducts are directly measured. The measured adducts might come from adducts removed from DNA and oxidative modification of deoxyribonucleotide in deoxyribonucleotide pool. Generally, the relationship between adducts in salivary DNA and tissues carrying with tumors must be clarified so that salivary DNA adducts can be taken as biological markers. The detection method of the invention provides a method of high accuracy and sensitivity and can be applied in clinical trial to find out meaningful disease biological markers.

The invention improves over the prior art and complies with patent application requirements, and thus is duly filed for patent application. While the invention has been described by device of specific embodiments, numerous modifications and variations could be made thereto by those generally skilled in the art without departing from the scope and spirit of the invention set forth in the claims.

1. A method of detecting DNA adduct in saliva comprising:
providing a salivary DNA (deoxyribonucleic acid);
adding at least one isotope-labeled internal standard and a plurality of enzymes into the salivary DNA to hydrolyze the salivary DNA into a plurality of nucleosides, wherein the isotope-labeled internal standards are [¹⁵N₅]AdG, [¹⁵N₅]CdG, [¹⁵N₅]εdAdo, [¹⁵N₃]εdCyd and [¹³C₁,¹⁵N₂],1,N²-εdGuo;
extracting the nucleosides by utilizing a solid-phase extraction column; and
detecting and quantifying at least one DNA adduct having one of 1,N²-propano-2′-deoxyguanosine derived from acrolein (AdG); 1,N²-propano-2′-deoxyguanosine derived from crotonaldehyde (CdG): 1,N⁶-etheno-2′-deoxvadenosine (εdAdo); 3,N⁴-etheno-2′-deoxvcytidine (εdCyd) and 1,N²-etheno-2′-deoxyguanosine (1,N²-εdGuo) with a quantification limit of 0.1 picogram, 0.5 picogram, 0.1 picogram, 0.5 picogram and 0.5 picogram, respectively, in the plurality of extracted nucleosides by utilizing a stable isotope dilution nanoflow liquid chromatography-nanospray ionization tandem mass spectrometry (LC-NSI/MS/MS).

2-4. (canceled)

5. The method of detecting DNA adduct in saliva as recited in claim 1, wherein the plurality of enzymes include micrococcal nuclease, phosphodiesterase II, adenosine deaminase and alkaline phosphatase.

6. The method of detecting DNA adduct in saliva as recited in claim 1, wherein the salivary DNA is at least 25 micrograms.

7. The method of detecting DNA adduct in saliva as recited in claim 1, wherein a spray voltage of the stable isotope dilution nanoflow LC-NSI/MS/MS is 1.3 to 2.0 kV.

8. The method of detecting DNA adduct in saliva as recited in claim 1, wherein a source temperature of the stable isotope dilution nanoflow LC-NSI/MS/MS is 200 to 300 degrees Celsius.

9. (canceled)
